(19) United States
(12) Patent Application Publication
(10) Pub. No.: US 2006/0241775 A1
(43) Pub. Date: Oct. 26, 2006

(54) Humeral joint prosthesis

(76) Inventor: Daniel D. Buss, Medina, MN (US)

Correspondence Address: Brooks & Cameron, PLLC, Suite 500, 1221 Nicollet Avenue, Minneapolis, MN, 55403, US

(21) Appl. No.: 11/398,909

(22) Filed: Apr. 6, 2006

Related U.S. Application Data
(60) Provisional application No. 60/673,634, filed on Apr. 21, 2005.

(51) Int. Cl. A61F 2/40

(52) U.S. Cl. 623/19.13

(57) ABSTRACT

Embodiments of the present invention relate to methods, devices, and systems for shoulder joint replacement. One embodiment of a humeral head prosthesis includes a first face for positioning against a surface of a glenoid, a second face for attachment to the stem, and an oval shaped circumference wherein a major axis is oriented between a top position and a bottom position and wherein the major axis is to be generally oriented in a superior-inferior orientation when positioned for use.

### CROSS-REFERENCE TO RELATED APPLICATIONS

This application claims the benefit of U.S. Provisional Application No. 60/673,634, filed Apr. 21, 2005, the entire content of which is incorporated herein by reference.

### BACKGROUND OF THE INVENTION

In the field of medicine, there are many situations that arise where a joint, such as a shoulder, knee, or hip, should be replaced. Each such joint contains an articulating surface that moves with respect to a support surface. For example, in the shoulder joint, one of the articulating surfaces is located on the head of the humerus bone (humerus). The head is often referred to as the humeral head.

The other articulating surface is located on the glenoid which forms a socket into which the humeral head is positioned and articulated. Surrounding the glenohumeral (“gleno” meaning of the glenoid and “humeral” meaning of the humerus) joint are a number of tendons and muscles that work together to articulate and stabilize the humerus. These tendons and muscles allow the shoulder to have an extensive range of motion.

As with any joint, overuse and age incurs wear and tear sometimes causing problems that can ultimately require a joint replacement. One condition that can result from an aging shoulder is Rotator Cuff Arthropathy (RCA).

RCA is an arthritis that develops in the shoulder after atrophy of the rotator cuff muscles allow the humerus to migrate upwards, or superiorly, making contact with the undersurface of another part of the shoulder, the acromion. This can create asymmetrical loading on the articular surface of the glenoid, for example. The displacement can create wear on the surface of the humeral head, the articular surface of the glenoid, and the acromion.

As a consequence, some RCA patients experience significant pain and shoulder dysfunction due in part to the superior migration of the head against the undersurface of the acromion. To alleviate pain and restore range of motion, a shoulder replacement, or, “arthroplasty” has historically been a solution to RCA.

A traditional total shoulder arthroplasty includes replacement of the humeral head and glenoid. In an arthroplasty procedure, the humeral head is dissected from the humeral stem and a rod with a spherical head is placed on the humeral stem. The damaged glenoid is reamed to a smooth surface to prepare for a polyethylene glenoid component to be cemented to the exposed glenoid.

When only the humeral head is replaced, the procedure is typically referred to as a hemiarthroplasty (i.e., “hemi” meaning half and “arthroplasty”). RCA patients typically have improved outcomes when receiving a hemiarthroplasty.

The replacement head in a traditional hemiarthroplasty is hemispherical in shape and is designed to articulate on the patient's existing glenoid. The humeral component is offered in various sizes depending on the patient's anatomy.

However, in the RCA patient, the traditional humeral head prosthetic can potentially cause problems. A hemiarthroplasty uses an intact rotator cuff which maintains the center of rotation of the humeral head on the glenoid. The RCA patient does not have an intact cuff, thus allowing the humeral head to migrate upwards, or superiorly, losing the necessary center of rotation as previously described.

In such instances, the patient can experience discomfort, pain, limited movement of the humerus, among other difficulties. In order to provide a comfortable amount and rate of articulation of the joint in RCA patients, oftentimes, replacement of the humeral head, or a portion thereof, with an oversized humeral head prosthesis can decrease the pain experienced. However, these oversized heads can, in some cases, be too large for the surrounding physiology and can result in painful and un-natural forces on the tendons and muscles surrounding the prosthetic head.

### BRIEF DESCRIPTION OF THE DRAWINGS

FIG. 1A illustrates an anterior/posterior view of a typical right shoulder.

FIG. 1B illustrates an anterior/posterior view of a right shoulder having RCA.

FIG. 1C illustrates an anterior/posterior view of a right shoulder with an embodiment of a humeral head of the present disclosure positioned on the end of a humerus.

FIG. 2A illustrates an axillary view of a typical glenoid/humeral joint.

FIG. 2B illustrates an axillary view of a glenoid/humeral joint having a prior art hemispherical implant.

FIG. 2C illustrates an axillary view of a glenoid/humeral joint having an embodiment of a humeral head implant of the present disclosure.

FIG. 3A illustrates an anterior/posterior view of a prosthetic humeral head mounted on a humerus.

FIG. 3B illustrates an axillary view of a prosthetic humeral head mounted on a humerus.

FIG. 3C illustrates a view of the humeral head taken along line 3C-3C of FIG. 3A.

FIG. 3D illustrates a top view of the humeral head taken along line 3D-3D of FIG. 3A.

### DETAILED DESCRIPTION OF THE INVENTION

Embodiments of the present invention provide a re-design of the traditional humeral head from a hemispherical shape to an elliptical shape that provides an increase in head size in the top to bottom, or superior to inferior, plane. Embodiments can also include a change in front to back, or anterior to posterior, plane. Such changes can better restore the center of rotation and deltoid lever arm muscle.

Embodiments of the present invention provide methods and humeral head prosthetic structures for use in shoulder joint replacement. Embodiments provide humeral head prostheses that have a head with a non-circular circumferential shape for placement in a superior-inferior orientation within the shoulder. In this way, the patient can have near normal to normal articulation in the superior-inferior plane.

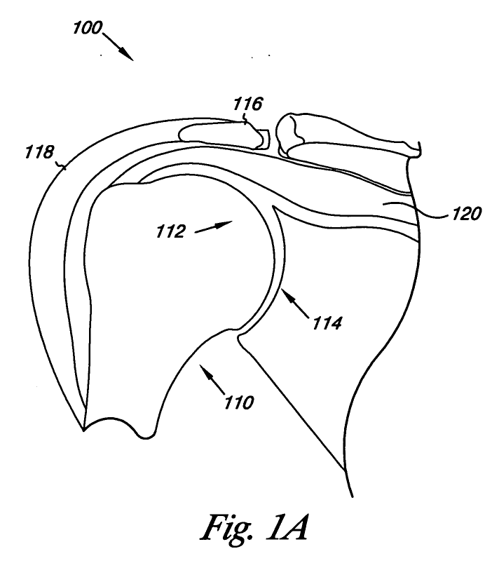

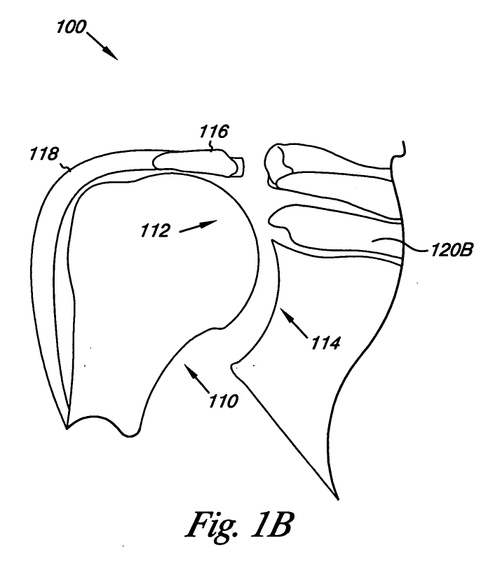

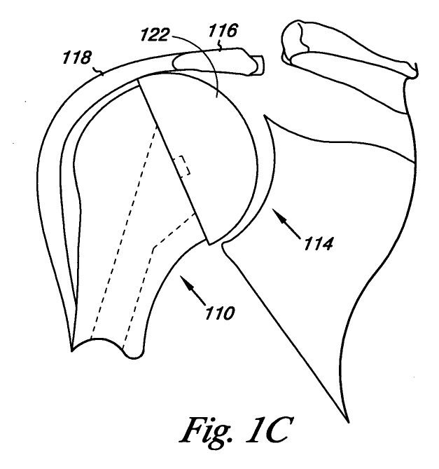

For the benefit of the reader, an example of a normal shoulder joint is shown in FIG. 1A with its component parts identified therein. FIG. 1A illustrates an anterior/posterior view of a shoulder joint 100 including a humerus 110 having a humeral head 112.

The humeral head 112 is positioned to articulate on a surface of the glenoid 114 which is part of the scapula. In this example, the interfacing surfaces of the humeral head 112 and the glenoid 114 are generally mating so as to provide smooth articulation of the humeral head 112. The shoulder joint 100 also includes a number of muscles and tendons, such as the rotator cuff (a portion of which is indicated at 120), that pass over the top of the humeral head 112 and under the acromion 116. The deltoid muscle 118 also works to move the humerus 110 upward.

In a patient with a rotator cuff deficient shoulder, the humeral head 112 can be forced upward over time, for example, by the pull from the deltoid. The result of this movement can be the wearing of the humeral head 112, the glenoid 114, and the acromion 116 and, in some cases, development of Rotator Cuff Arthropathy (RCA). For example, as shown in FIG. 1B, the head has moved upward, thereby wearing the upper portion of the glenoid 114 away.

FIG. 1B illustrates a shoulder having RCA which, as discussed above is an arthritis that develops in the shoulder after certain types of rotator cuff tears. Generally, the rotator cuff includes the supraspinatus, infraspinatus, teres minor, and subscapularis muscles.

These muscles, and their associated tendons, are interposed between the humerus and the scapula and work together to articulate and stabilize the humerus when it moves, thereby maintaining the position of the center of rotation of the glenoid and humerus. A rotator cuff tear can include the tearing of one or more of these muscles. For example, as shown in FIG. 1B, the rotator cuff 120B represents a retracted and atrophied supraspinatus.

In some instances, a rotator cuff tear allows the humerus to migrate upward which can lead to unusual loading of the glenoid and can result in abnormal articular wear to the humeral head, the glenoid, and the acromion. Many such patients experience significant pain and shoulder dysfunction.

For example, in the shoulder illustrated in FIG. 1B, through articulation of the humeral head 112, the upper portion of the humeral head has been worn through its contact with the acromion which has also been worn such that its shape has changed. This interaction can create significant pain and/or limited range of motion to the patient.

In many instances, a hemispherical prosthetic head may be used, but since the interface with the humeral head and the glenoid in such cases is directing articulation forces on a smaller portion of the humeral head and glenoid surfaces, damage to the humeral head and, more likely, the surface of the glenoid can continue.

Prosthetic heads having the shape of a hemisphere of an oblate spheroid (circular circumference, but squashed articulating surface) allow for greater contact with the glenoid, since the articulation surface is larger and since the articulation surface is flatter. However, since the circumference is circular, in order to provide a prosthetic head with the correct central point of articulation, the circumference of the head has to be oversized.

The over-sizing of the prosthetic head can be such that it pushes on the muscles and tendons surrounding the shoulder joint on the anterior and posterior portions of the joint. Such interaction with the tendons and muscles can cause bulging, wearing, and tearing of the muscles and tendons surrounding those portions, among other issues.

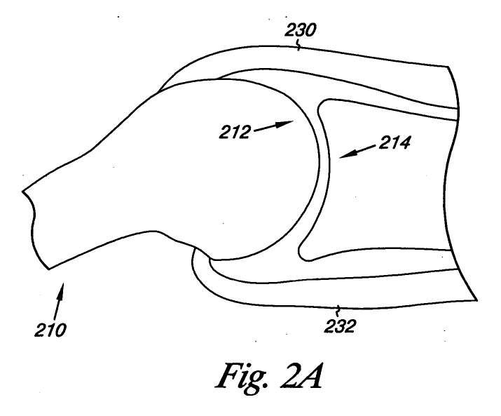

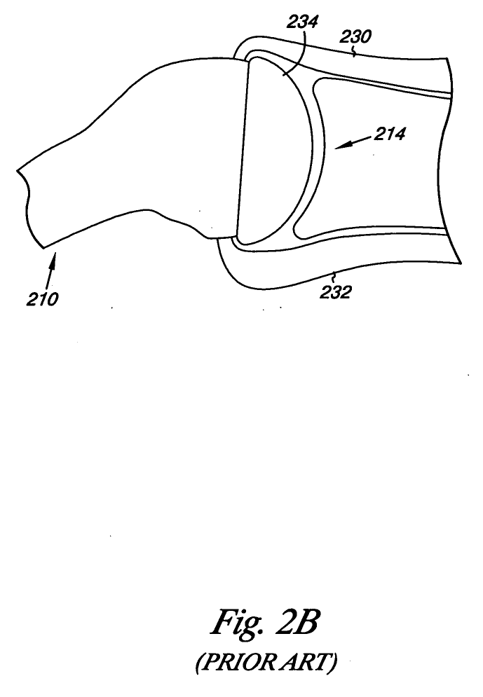

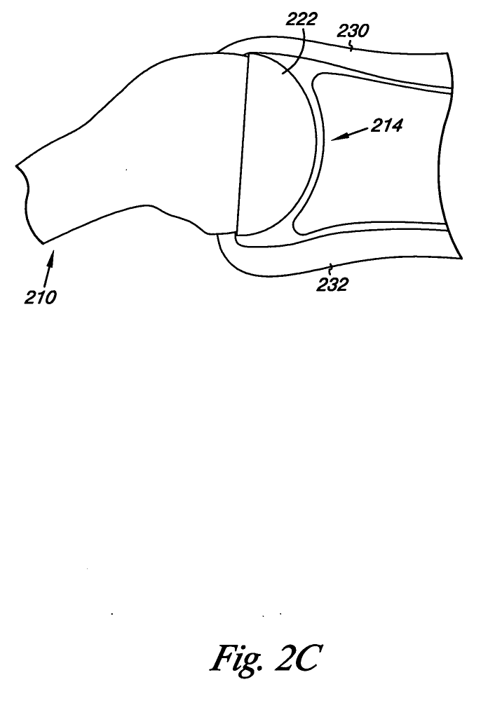

FIG. 1C illustrates an embodiment of the present disclosure wherein a humeral head prosthesis 122 has been placed within the right shoulder joint of a patient. This illustration shows that a traditional hemiarthroplasty does not typically restore the center of rotation, particularly in RCA patients. In this embodiment, the humeral head 122 has an oval shaped circumferential shape. Such prosthetic humeral heads are discussed in more detail with respect to FIGS. 2A-2C.

An embodiment of a humeral head prosthetic is illustrated in FIGS. 2A-2C. FIG. 2A illustrates an axillary view of a typical glenoid/humeral joint. As shown in this figure, the glenoid 214 and humeral head 212 of the humerus 210 have generally mating surface that engage during articulation of the joint. These two surfaces are maintained proximal to each other, or in contact with each other, by muscles of the rotator cuff.

In this illustration, two such muscles are shown, the subscapularis 230 and the teres minor 232. This view of the joint is typically not significantly different when an RCA condition has developed. However, as is shown in FIG. 2B, in some cases, when a regular hemispherical head (i.e., a half sphere) is implanted, the head may not provide a proper fit within this space.

FIG. 2B illustrates an axillary view of a glenoid/humeral joint having a prior art hemispherical implant. As can be seen in this figure, the hemispherical head 234 mounted on the humerus 210 can cause tensioning of the subscapularis 230 and teres minor 232, thereby potentially causing discomfort and damage to the muscles of the rotator cuff and other components of the joint, such as the glenoid 214.

FIG. 2C illustrates an axillary view of a glenoid/humeral joint having an embodiment of a humeral head implant of the present disclosure. In various embodiments of the present disclosure, the reduced anterior/posterior width of the humeral head implant 222 attached to the humerus 210 provides a better fit within the space provided.

In the embodiment illustrated in FIG. 2C, the tension on the subscapularis 230 and teres minor 232 can be reduced or eliminated while maintaining a center of rotation on the glenoid 214. In some embodiments, the surface of the head 222 in the anterior/posterior dimension can have a more arcuate shape, as shown in the embodiment of FIG. 2C. In this way, the articulation surface of the humeral head 222 can be a better fit than a hemispherical head implant, such as that shown in FIG. 2B.

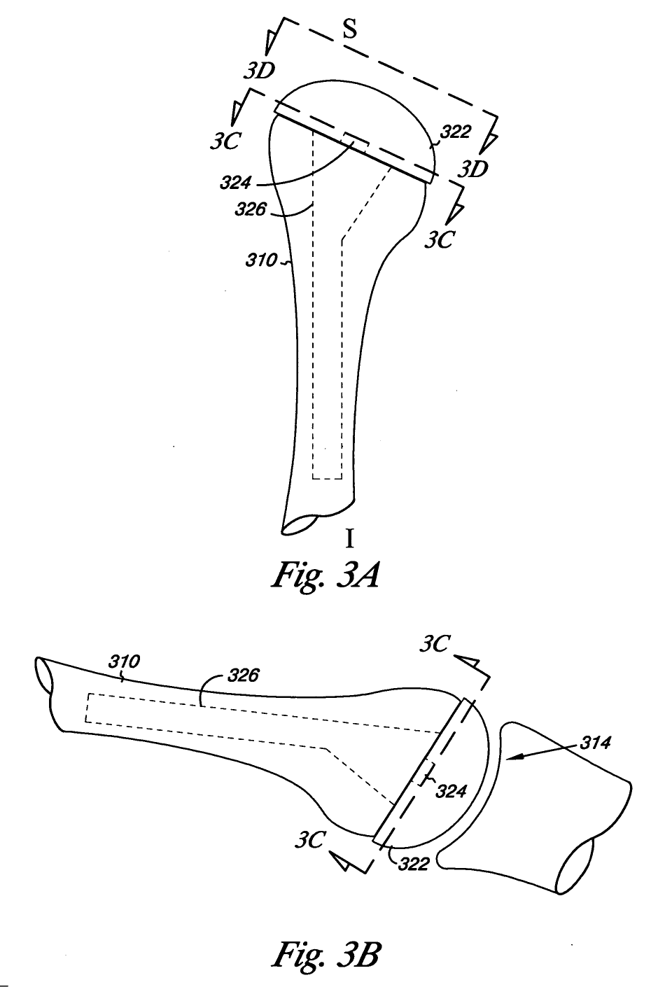

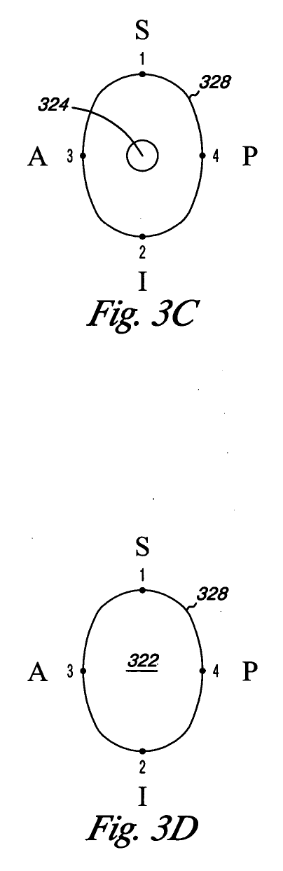

FIGS. 3A-3D illustrate the shape of a humeral head implant embodiment of the present disclosure. Specifically, FIG. 3A illustrates an anterior/posterior view of a prosthetic humeral head mounted on a humerus. FIG. 3B illustrates an axillary view of a prosthetic humeral head mounted on a humerus. FIG. 3C illustrates a view of the humeral head taken along line 3C-3C of FIG. 3A. FIG. 3D illustrates a top view of the humeral head taken along line 3D-3D of FIG. 3A.

The shape of the prosthesis illustrated in FIG. 3A-3D, is that of roughly a hemisphere of an ellipsoid (i.e., a half of an ellipsoid). An ellipsoid can be defined as a three dimensional object that is produced by rotating an ellipse (i.e., a two dimensional object) about one of its axes.

In the case of the ellipsoid hemisphere illustrated in FIG. 3A-3D, the ellipsoid shape is that based upon the rotation of an ellipse around its axis of elongation. Although an ellipsoid shape is shown in this embodiment, other oval shapes can be used as are described in more detail below.

In various embodiments, including the embodiment shown in FIGS. 3A-3D, the humeral head prosthesis 322 has a first face for positioning against a surface of a glenoid, a second face for attachment to the stem of the humerus 310, and an oval shaped circumference 328 (shown in FIGS. 3C and 3D) wherein a major axis (i.e., the axis between points 1 and 2 in FIGS. 3C and 3D) is oriented between a top position and a bottom position.

In such embodiments, the major axis is to be generally oriented in a superior-inferior orientation when positioned for use (e.g., indicated by the S and I symbols in FIGS. 3A-3D). In this way, the larger dimension of the humeral head can be used for articulation of the humerus 310 in generally upward and downward movement. For example, the relationship of the surface of the humeral head and the glenoid can be seen in FIG. 3B, wherein the articulation surface of the humeral head and the surface of the glenoid provide a mating relationship that can reduce wear on the various components of the joint.

The shape can, in some instances, also allow for the greater surface area interaction between the surface of the prosthetic humeral head 322 and the glenoid 314. In this way, the force used for articulation and other forces placed on the humeral head can be applied over a larger area of the humeral head and the surface of the glenoid. This can reduce damage to the humeral head and glenoid, among other parts of the shoulder.

Further, since the elongate direction of the prosthetic humeral head is provided in the superior-inferior direction, in some cases, the prosthetic humeral head can be positioned lower than the original humeral head, for example, due to the reduction of force applied to the upper portion of the surface of articulation of the humeral head.

In the embodiment shown in FIG. 3A, the humeral head has an elliptical circumference. Specifically, the humeral head has a shape of a half of an ellipsoid. In some embodiments, the humeral head can have a Cartesian oval shaped circumference. The Cartesian oval shape is similar to that of an egg shape.

Other shapes that can be implemented include, but are not limited to various oval shapes such as ellipses, and hemispherical shapes. For example the surface of articulation can have a half ellipse shaped cross-section, a half oblate hemisphere shaped cross-section, or a half prolate hemisphere shaped cross-section, among others.

In various embodiments, a shoulder prosthesis can include a stem having a distal end for insertion in the superior end of a humerus. For example, as shown in FIG. 3A, the embodiment includes a stem 326 for insertion into the humerus 310.

In such embodiments, the stem can be attached to the humeral head prosthesis in any manner. For example, the prosthetic humeral head can be uniformly fabricated with the stem, can be welded or otherwise adhered to the stem, can be threaded onto the stem, and other such methods.

The connection can include a bore in the second face of the prosthetic humeral head. For example, the embodiment of FIG. 3A includes a bore 324 formed in the bottom surface of the prosthetic humeral head 322. In such embodiments, the stem can include a shaft sized to fit within the bore 324.

The shaft can be secured to the bore in any manner. For example, the shaft can be threaded and mating threads can be formed on the interior surface of the bore to secure the bore and shaft together, among other fastening mechanisms.

In some embodiments, the shaft can be on the prosthetic humeral head and the bore can be on the stem. The connection of the prosthetic humeral head and the stem can be positioned non-coaxially with a central axis of the center of the prosthetic humeral head, and/or the axis of elongation of the stem. For example, the attachment point of the stem and prosthetic humeral head is non-coaxial with respect to the axis elongation of the stem.

Additionally, the stem 326 can be attached to the humerus 310 in any manner. For example, the embodiment shown in FIG. 3A is a Morse taper design which takes advantage of the Morse taper principle in which the slight tapering of the sides of the stem inward from the top of the stem 326 to the bottom act to frictionally hold the stem within the aperture created in the humerus 310. Specifically, the Morse taper principle can be defined as being tapered to effect a strong mechanical connection after being impacted into a receptacle of corresponding geometric form, in this case, the hollowed out interior of the humerus.

Embodiments also include various shoulder prosthesis replacement kits having a stem with a distal end for insertion in the superior end of a humerus, a humeral head including a first face for positioning against a surface of a glenoid, and a second face for attachment to the stem. In such embodiments, the humeral head can have an oval shaped circumference. The major axis of the humeral head, in such embodiments, can be oriented between a top position and a bottom position. Kits can also include a prosthetic glenoid having a surface for positioning against the first face of the humeral head.

In such embodiments, the surface of the glenoid, for positioning against the first face of the humeral head, can have a mating or complimentary shaped surface. Such surface can allow for better sliding engagement between the articulating surface of the prosthetic humeral head and the engagement surface of the glenoid.

In some embodiments, a surface that is complimentary may be a suitable shape for interfacing with the articulation surface of the prosthetic humeral head, but may not be a surface that has a mating relationship with the articulation surface. For example, the articulation surface may have a curved surface with a different angle of curvature than that of the engagement surface of the glenoid.

Such embodiments may provided greater freedom of movement of the humerus with respect to the glenoid and may allow for the contact point between the glenoid and the humeral head to change, thereby reducing wear on a particular location on the humeral head and/or the glenoid.

Further, the circumference of the glenoid can be a shape that can be matingly oriented with the surface of the articulating surface of the prosthetic humeral head. For example, in some embodiments, the glenoid can include an oval edge. The oval edge can, for example, include a major axis that is oriented in a superior-inferior orientation.

The surface of the glenoid for positioning against the first face of the humeral head can also be an elliptical edge. In such embodiments, the elliptical edge can have a major axis that is oriented in a superior-inferior orientation.

Embodiments also include methods for replacing a humeral head in a patient. For example, methods can include attaching a prosthetic humeral head to a humerus of a patient, the prosthetic humeral head including a first face for positioning against a surface of a glenoid, and a second face, where the head has an oval shaped circumference and a major axis that is oriented between a top position and a bottom position.

Methods can also include attaching a prosthetic glenoid surface to a glenoid of a patient, positioning the prosthetic humeral head and the prosthetic glenoid in an adjacent orientation with respect to each other, and/or repairing surrounding tissue to encapsulate the prosthetic humeral head and glenoid. Attaching the prosthetic humeral head to the humerus of the patient can include attachment of the prosthetic humeral head to the stem. Method embodiments can also include inserting a stem into a cavity formed in the humerus.

Although specific embodiments have been illustrated and described herein, those of ordinary skill in the art will appreciate that an arrangement calculated to achieve the same techniques can be substituted for the specific embodiments shown. As one of ordinary skill in the art will appreciate upon reading this disclosure, various embodiments of the invention can be performed in one or more devices, device types, and system environments including networked environments.

Combination of the above embodiments, and other embodiments not specifically described herein will be apparent to those of skill in the art upon reviewing the above description. The scope of the various embodiments of the invention includes other applications in which the above structures and methods can be used. Therefore, the scope of various embodiments of the invention should be determined with reference to the appended claims, along with the full range of equivalents to which such claims are entitled.

In the foregoing Detailed Description, various features are grouped together in a single embodiment for the purpose of streamlining the disclosure. This method of disclosure is not to be interpreted as reflecting an intention that the embodiments of the invention require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A humeral head prosthesis, comprising;
a first face for positioning against a surface of a glenoid;
a second face for attachment to the stem; and
an oval shaped circumference wherein a major axis is oriented between a top position and a bottom position and wherein the major axis is to be generally oriented in a superior-inferior orientation when positioned for use.

2. The prosthesis of claim 1, wherein the humeral head has an elliptical circumference.

3. The prosthesis of claim 1, wherein the humeral head has a shape of a half of an ellipsoid.

4. The prosthesis of claim 1, wherein the humeral head has a Cartesian oval shaped circumference.

5. The prosthesis of claim 1, wherein the first face has a half ellipse shaped cross-section.

6. The prosthesis of claim 1, wherein the first face has a half oblate hemisphere shaped cross-section.

7. The prosthesis of claim 1, wherein the first face has a half prolate hemisphere shaped cross-section.

8. A shoulder prosthesis, comprising:
a stem having a distal end for insertion in the superior end of a humerus; and
a humeral head including;
a first face for positioning against a surface of a glenoid; and
a second face for attachment to the stem, wherein the head has an oval shaped circumference wherein a major axis is oriented between a top position and a bottom position.

9. The prosthesis of claim 8, wherein the prosthesis further includes a flange protruding from at least one of the first and second faces.

10. The prosthesis of claim 9, wherein the flange protrudes at an angle with respect to the orientation of the second face.

11. The prosthesis of claim 8, wherein the prosthesis further includes a flange protruding out of the second face.

12. The prosthesis of claim 11, wherein the flange is positioned at a junction between the first face and the second face.

13. The prosthesis of claim 8, wherein the second face is designed to mate with a first surface of the humerus and wherein the prosthesis further includes a flange constructed and arranged to mate with a second surface of the humerus.

14. A shoulder prosthesis, comprising:
a stem having a distal end for insertion in the superior end of a humerus;
a humeral head including a first face for positioning against a surface of a glenoid, and a second face for attachment to the stem, wherein the head has an oval shaped circumference wherein a major axis is oriented between a top position and a bottom position; and
means for connecting the head to a proximal end of the stem.

15. The prosthesis of claim 14, wherein the means for connecting includes a bore in the second face of the humeral head.

16. The prosthesis of claim 14, wherein the means for connecting includes a shaft on the second face of the humeral head.

17. The prosthesis of claim 14, wherein the means for connecting is positioned non-coaxially with a central axis of the first face.

18. The prosthesis of claim 14, wherein means for connecting includes a protruding shaft on the stem having a Morse taper.

19. The prosthesis of claim 14, wherein means for connecting includes a protruding shaft on the second face having a Morse taper.

20. A shoulder prosthesis replacement kit, comprising;
a stem having a distal end for insertion in the superior end of a humerus;
a humeral head including a first face for positioning against a surface of a glenoid, and a second face for attachment to the stem, wherein the head has an oval shaped circumference wherein a major axis is oriented between a top position and a bottom position; and
a glenoid having a surface for positioning against the first face of the humeral head.

21. The prosthesis replacement kit of claim 20, wherein the surface of the glenoid for positioning against the first face of the humeral head has an oval edge.

22. The prosthesis replacement kit of claim 21, wherein the oval edge has a major axis and wherein the major axis is oriented in a superior-inferior orientation.

23. The prosthesis replacement kit of claim 20, wherein the surface of the glenoid for positioning against the first face of the humeral head has an elliptical edge.

24. The prosthesis replacement kit of claim 23, wherein the elliptical edge has a major axis and wherein the major axis is oriented in a superior-inferior orientation.

25. The prosthesis replacement kit of claim 20, wherein the surface of the glenoid for positioning against the first face of the humeral head is a complimentary surface.

26. The prosthesis replacement kit of claim 20, wherein the surface of the glenoid for positioning against the first face of the humeral head is a mating surface.

27. A method of replacing a humeral head in a patient, comprising:
attaching a prosthetic humeral head to a humerus of a patient, the prosthetic humeral head including a first face for positioning against a surface of a glenoid, and a second face, wherein the head has an oval shaped circumference wherein a major axis is oriented between a top position and a bottom position;
attaching a prosthetic glenoid surface to a glenoid of a patient;
positioning the prosthetic humeral head and the prosthetic glenoid in an adjacent orientation with respect to each other; and
repairing surrounding tissue to encapsulate the prosthetic humeral head and glenoid.

28. The method of claim 27, wherein the method further includes inserting a stem into a cavity formed in the humerus.

29. The method of claim 28, wherein attaching the prosthetic humeral head to the humerus of the patient includes attachment of the prosthetic humeral head to the stem.
